(19) United States
(12) Patent Application Publication
(10) Pub. No.: US 2006/0075922 A1
(43) Pub. Date: Apr. 13, 2006

(54) Controlled-acidity composition

(76) Inventors: Albert P. Supik, Towson, MD (US); Frank Gigliotti, Phoenix, MD (US)

Correspondence Address: ROYAL W. CRAIG, SUITE 153, 10 NORTH CALVERT STREET, BALTIMORE, MD, 21202, US

(21) Appl. No.: 11/213,439

(22) Filed: Aug. 26, 2005

Related U.S. Application Data
(60) Provisional application No. 60/604,511, filed on Aug. 26, 2004.

(51) Int. Cl. C09D 5/14; C09C 1/02

(52) U.S. Cl. 106/15.05; 106/464

(57) ABSTRACT

A stabilized acidic composition comprised essentially of two core components that are GRAS and can be safely used in foodstuffs, and yet which provide a medium that destroys microbes and/or can regulate pH safely and effectively. Two core components include a first inorganic acid, such as hydrochloric or sulfuric acid, that dissociates nearly to completion in water, and a second organic GRAS acid, such as organic hydroxy acid or citric acid. The stabilized acidic composition serves as an effective antimicrobial solution or a low pH medium while maintaining a larger margin of safety than is conventionally known. Thus, the stabilized acidic composition of the present invention is safe for industrial, medical, agricultural and military applications that require the need for a low pH environment that is not harmful to human or animal skin, and can be used in processing or manufacturing of ingestibles.

### CROSS-REFERENCE TO RELATED APPLICATIONS

The present application derives priority from U.S. Provisional Patent Application No. 60/604,511 for “CONTROLLED-ACIDITY COMPOSITION”; Filed: Aug. 26, 2004.

### FIELD OF THE INVENTION

The present invention relates to acid compositions that are useful in pH control and antimicrobial activity for various products used in commercial industries such as food processing, agricultural, and preservation that are used to inhibit the growth of microorganisms and, more particularly, to a stabilized acid composition antiseptic that is safe for contact with humans and animals, and are safe for use in food and beverages.

### BACKGROUND OF THE INVENTION

Acidic solutions that contain various acid compositions have been used for cleaning and disinfecting a variety of household and medical surfaces, and also to control the pH for various processing applications that require stringent pH levels to be maintained. The effectiveness of such products relies a low pH level which effects a reactive milieu or the direct inactivation of microbes. Unfortunately, many conventional solutions are harmful to the user if the product comes in contact with the skin, or if the user inhales fumes from the product, or ingests the product.

Many of the acidic compositions currently used in industrial, medical, military and household settings present health hazards to the user and are corrosive with prolonged exposure to the surfaces they contact. Use of these compositions often requires special protective clothing and application methods. Additionally, disposal of these toxic products in a manner which ensures the safety of the environment and personnel is costly and time consuming. Although some conventional compositions are less caustic to skin and, overall, less hazardous to the user than others, they are, nonetheless, not safe for use in food and beverages or in other ingestible products.

Examples of the various existing multiple acid compositions include U.S. Pat. No. 4,477,364 to Garcia which discloses an acidic glass cleaning composition comprising from about 1 to about 15 weight percent hydrofluoric acid and from about 85 to about 99 weight percent of an aqueous acidic composition having a pH value of less than about 1. The acidic constituent is a mix of from about 20 to about 30 weight percent hydrochloric acid with from about 16 to about 26 weight percent phosphoric acid, and the entire mix is diluted in water.

U.S. Pat. No. 4,483,887 to Garcia discloses a plating solution for metal plating comprising from about 1 to about 35 weight percent of an aqueous solution containing metal ions to be deposited on the substrate, and from about 65 to about 99 weight percent of an aqueous acidic component having a pH value of less than about 1. The aqueous acidic component includes 45 to about 85 weight percent hydrochloric acid with from about 20 to about 55 weight percent phosphoric acid, an effective amount of water, about 2 to about 20 weight percent of a hydroxy carboxylic acid and from about 0 to about 20 weight percent of a dicarboxylic acid.

Mr. Garcia also discloses in his U.S. Pat. No. 4,970,014 an acidic solution for use as an aluminum cleaning and brightening including about 1-15 weight percent hydrofluoric acid, about 1-5 weight percent sulfuric acid, and about 1-5 weight percent phosphoric acid, and in his U.S. Pat. No. 5,019,288 an acidic solution for use as a cleaning composition for copper and copper alloys including U.S. Pat. No. 4,675,120 to Martucci discloses acidic compositions of hydrochloric, phosphoric, oxalic and citric acids, optionally additionally containing oleic, hydrofluoric or sulfuric acid and methods of preparing the acidic mixtures. The compositions are useful for well acidizing, cleaning glass, in batteries, removing corrosion from metals, removing oil residues from oil handling equipment and reactivating wells clogged with asphaltene.

While the foregoing acid solutions may be effective in their claims, they will still cause damage, burning, or reddening to the skin and human tissues. Therefore, these formulations cannot be used when skin contact is possible, and certainly not for use in ingestible products.

One additional acid composition described in U.S. Pat. No. 5,512,200 to Garcia is reported to be non-irritating to human skin and therefore can be used in products such as cleansers and topical pharmaceuticals. However, one of the components is not considered to be “generally recognized as safe” (GRAS) and therefore cannot be used in ingestible products, or in products that come in contact with foodstuffs.

Another acidic composition disclosed in U.S. Pat. No. 375,976 to Roden et al. is said to be a safe and effective low pH composition that contains phosphoric acid as a core ingredients. While this composition is “generally recognized as safe” (GRAS) and can ostensibly be used in ingest able the FDA as safe for use in foods, beverages and ingestible products, and in products associated with foods and beverages. Such substances are generally considered non-carcinogenic.

It would be far more advantageous to provide an acidic composition for various industrial, agricultural and food processing processes that requires a low pH environment, and is stabilized in its ability to avoid damage to human or animal skin while providing an acidic environment inhibits microbial growth, and which is of no harm to the environment. The present inventors have developed acidic compositions which, in their preferred embodiment, essentially comprise two components that are GRAS and can be safely used in foodstuffs such as soft drinks, and yet which provide a medium that destroys microbes and/or can regulate pH safely and effectively.

### SUMMARY OF THE INVENTION

Accordingly, it is an object of the present invention to provide acid compositions that are useful in pH control and antimicrobial activity for a range of products used in commercial industries such as food processing, agricultural, and preservation.

It is another object to provide acid compositions as described above that stabilized and completely safe for contact with humans and animals, and for use in food and beverages.

It is another object to provide a safe and effective low pH composition as described above in which all constituents are “generally recognized as safe” (GRAS) and can be used in ingest able products, with no environmental problems from runoff.

According to the present invention, the above-described and other objects are accomplished by providing a stabilized acidic composition comprised essentially of two core components that are GRAS and can be safely used in foodstuffs such as soft drinks, and yet which provide a medium that destroys microbes and/or can regulate pH safely and effectively. The stabilized acidic composition serves as an effective antimicrobial solution or a low pH medium while maintaining a larger margin of safety than is conventionally known. Thus, the stabilized acidic composition of the present invention is safe for use as either the sole or major component of solutions including, but not limited to, disinfectants, cleansers and cosmetic agents, packaging and wrapping of food, food containers, and shelf-life extenders. The invention can also be used for industrial, agricultural, medical, and military applications that require the need for a low pH environment that is not harmful to human or animal skin, and can be used in processing or manufacturing of ingestible.

In a preferred embodiment, the two core components that are GRAS include a first inorganic acid, such as hydrochloric or sulfuric acid, that dissociates nearly to completion in water; and a second organic GRAS acid, such as organic hydroxy acid or citric acid.

### DETAILED DESCRIPTION OF THE PREFERRED EMBODIMENTS

The present invention is a stabilized acidic composition comprised essentially of two core components plus water, the core components being GRAS and can be safely used in foodstuffs such as soft drinks, the mix providing a medium that destroys microbes and/or can regulate pH safely and effectively. In a preferred embodiment, the two core components that are GRAS include a first inorganic acid that dissociates nearly to completion in water; and a second organic GRAS acid.

In one embodiment, the first inorganic acid may be hydrochloric acid provided in a range between 0.01 to about 35 percent or sulfuric acid provided in a range between 0.01 to about 60 weight percent of the final composition. Hydrochloric acid (CAS# 7647-01-0), or hydrogen chloride (HC L) is a colorless to pale yellow liquid with a pungent odor, sulfuric acid (CAS# 7664-93-9), or hydrogen sulfate (HS2S04) is a colorless to slightly brown liquid. The Food and Drug Administration has listed both hydrochloric acid and sulfuric acid as GRAS when used in accordance with good manufacturing practices. The second organic acid may be citric acid and is provided in a range of between 4.95 to about 54 weight percent of the final composition. Citric acid is the most widely used food acidulant, and is commonly produced through a sucrose fermentation technique using Aspergillus niger. The citric acid is recovered by precipitation, evaporation or crystallization. It is available in two forms: monohydrate or anhydrous (approximately 8.6% moisture), and is available as a solution. The product may also be obtained by naturally occurring acids. Citric acid is a white powder or colorless translucent crystal. It is odorless with a strong acidic flavor that rapidly dissipates on the palate. One gram is soluble in 0.5 ml water. The pH of a 1% solution of the monohydrate is 2.3 and of a 1% solution of the anhydrous form is 2.2. The Food and Drug Administration has listed citric acid as GRAS when used in accordance with good manufacturing practice.

The above-described core components are diluted with water as described below.

The stabilized acidic composition described above is prepared in accordance with the following steps:

1. Mix at room temperature a solution of about 5 to about 60 weight percent citric acid, and most preferably 20 weight percent, with potable water for a period of time sufficient to result in thorough mixing;

2^{nd}. Add by stirring at room temperature either an aqueous solution of hydrochloric acid of 0.01 to 35 percent or sulfuric acid of 0.01 to 60 percent to the citric acid water mix to produce a homogeneous blend.

When prepared in accordance with the foregoing method, the acid aqueous composition of the present invention is colorless (high concentrations of HCl produce a greenish to yellowish color), has a pH of less than one to 4, will not harm human or animal skin, and contains only substances approved by the Food and Drug Administration to be GRAS substances. Thus, the resulting aqueous acidic composition is safe for use in food, drink, and other ingestible products.

The composition of the present invention is also much less corrosive to metals than acid compositions at a similar pH.

Other GRAS organic acids may be substituted for citric acid, including acetic, adipic, fumaric, lactic, malic and tartaric, and ascorbic acid. The acid composition prepared as described above may be used alone or as the base or active component in the formulation of other solutions. Additional ingredients, if desired, may be added to the two-acid composition depending on the intended purpose or application. For example, other aerosols, dyes, alcohols, reducing agents, anesthetic agents, oxidizing agents, amines, amides, surfactants, creams, gels and other acids may be added to the compositions of the present invention as needed for a particular application.

The compositions of the present invention are suitable for use in a plurality of food industry, household, military, medical, agricultural and industrial settings. For example, potential uses of the compositions of the present invention include: cleaning and disinfecting surfaces, instruments, foods and equipment; antimicrobial component for hygiene products, preparation of skin surface for injections or surgical preparations, topical ointments, creams, gels, inhalants, mouth and eye washes, activation of immune-response (e.g. stimulates non-specific immunity); anti-odorant; cleaning and disinfecting food items and food processing (e.g. removes coffee and tea stains), packaging and storage materials; pH- and/or microbial-control agent (swimming pools); detoxifying composition for industrial settings (i.e. paper mills and other industrial plants, and laboratories may use HCl and/or sulfuric acids; can treat HCl burns with invention); industrial showers (acid showers): manufacture of acid-containing solutions and products (i.e. batteries containing “battery acid” such as sulfuric acid; by mixing with invention, less toxic and less fuming); cleaning battery posts; sterilization or purification of water supply;
non-toxic embalming agent; detoxification/deactivation of chemical and biological warfare agents; and cleaning air gels, condoms, mouthwashes, douches); treatment of burns, sunburns, ear infections, insect bites, jellyfish stings, anti-coagulant, treatment of medical waste, and anti-fungal agents (e.g. anti jock itch treatment, prevention or treatment of athlete's foot or ringworm).

One particular embodiment of the invention is an antimicrobial agent such as a disinfectants or sterilants used, for example, to sterilize drinking water (bottled or tap), disinfect surfaces, treat wounds, sterilize hair care and manicure equipment, sterilize dental equipment, sterilize hospital clean rooms, sterilize tissue culture hoods in laboratories, and sterilize biological waste. The acid composition of the present invention may also be used in cleaning or sterilizing containers used in the transportation and storage of food and drink, such as truck tanks, vats and fluid lines. In this context, the efficacy of the present invention has been demonstrated as per the following examples, which should not be viewed as limiting the scope of the invention.

### EXAMPLE 1

### Demonstration of Antimicrobial Activity (Staphylococcus aureus)

A typical antimicrobial solution can be prepared as described above by first dissolving the solid citric acid in potable water, and then adding the desired amount of hydrochloric acid such that the final solution has a pH of less than 3. A Staphylococcus aureus strain was obtained from a commercial of the mixture was placed into 3 volumes of fresh nutrient broth, and allowed to incubate at room temperature. Bacterial growth was assessed by measuring the density of the bacteria in nutrient broth solution at 750 nm in a spectrophotometer at various time points. An increase in density was assumed to indicate bacteria growth, and no change in density was assumed to indicate that the bacterial cells were not alive. The following table charts the results:

| | 0 minutes | 30 minutes | 60 minutes |
|---|---|---|---|
| Control (water) | 0.55 | 0.89 | 1.40 |
| 50% test solution | 0.42 | 0.41 | 0.45 |
| 10% test solution | 0.56 | 0.54 | 0.55 |
| 1% test solution | 0.47 | 0.52 | 0.66 |

It should be apparent that the invention kills this bacteria.

### EXAMPLE 2

### Demonstration of Antimicrobial Activity (Escherichia coli)

An Eshericia coli strain was obtained from a commercial source. The bacteria were inoculated and grown for 16 hr at room temperature in sterile nutrient broth. One volume of the bacteria was placed into 1 volume of either potable water or one volume of test solution for 10 minutes. The test solution was comprised of various concentrations of the low pH composition of the current invention.

After 10 minutes of incubation at room temperature, one volume of the mixture was placed into 3 volumes of fresh nutrient broth, and allowed to incubate at room temperature. Bacterial growth was assessed by measuring the density of the bacteria in nutrient broth solution at 750 nm in a spectrophotometer at various time points. An increase in density was assumed to indicate bacteria growth, and no change in density was assumed to indicate that the bacterial cells were not alive.

| | 0 minutes | 30 minutes | 60 minutes |
|---|---|---|---|
| Control (water) | 0.44 | 0.78 | 1.22 |
| 50% test solution | 0.21 | 0.22 | 0.21 |
| 10% test solution | 0.32 | 0.29 | 0.28 |
| 1% test solution | 0.40 | 0.39 | 0.41 |

Again, it can be concluded that the invention kills this bacteria.

### EXAMPLE 3

### Demonstration of Antimicrobial Activity (Camphylobacter jejuni)

A Camphylobacter jejuni strain was obtained from a commercial source. The bacteria were inoculated and grown in commercial media for this strain, and incubated under special gaseous conditions as provided by the company, BioControl. Growth plates for the bacteria were obtained from this same source. One volume of the grown bacteria was placed into 1 volume of either potable water or one volume of test solution for 10 minutes. The test solution was comprised of various concentrations of the low pH composition of the current invention. After 10 minutes of incubation at room temperature, one volume of the mixture was serially diluted and 0.1 mL volumes were placed onto the growth plates. The growth plates were incubated overnight, and the number colonies that appeared on the plates were determined by visual inspection.

| | 11/10 colonies | 1/100 colonies | 1/1000 colonies |
|---|---|---|---|
| Control (water) | >400 | 354 | 64 |
| 50% test solution | 4 | 0 | 0 |
| 10% test solution | 8 | 2 | 2 |
| 1% test solution | 12 | 4 | 2 |

Once again, it can be concluded that the invention kills this bacteria.

In addition to the foregoing, the composition can be used as an anti-odorant to neutralize ammonia-based odors/wastes, biological specimens, chemical toilets, animal bedding and diapers. It may be used as an underarm deodorant. In connection with food, it may be used to spray produce, clean and disinfect food transport containers and fluid lines or any surface coming in contact with food and food serving materials. It may be applied to seafood as a deodorizer and be used to spray live animals or to spray meat before wrapping, etc. As an antibacterial, anti-fungal and sporicide, it may be used, for example, as an acidifier in home canning.

With respect to military applications, the invention may be used to decontaminate chemical warfare agents on personnel and surfaces, and given its broad spectrum of activity, is ideal for incorporation into bandages and sponges. The invention may be deployed via missile or other airborne delivery device as a countermeasure to deactivate chemical warfare or biological agents (e.g. protein toxins such as anthrax, botulism and E. coli) delivered by or contained in another weapon system. It may be delivered to a broad contaminated area through the use of a fog/smoke generating device, crop-dusting or firefighting aircraft. Applications also include use as a non-toxic embalming agent, anti-scale build-up and treatment of water supplies, electrolyte sports drink, treatment of personal items such as toothbrushes or hairbrushes, safety showers for certain industries using acids, acid spill or acid cloud clean up. It may be used as a silver or chrome polish, to remove oxidation build-up on heat exchangers, pipes and water heaters, to remove scale from sinks, water storage tanks, showers and the like to remove barnacles, or to clean concrete. It may be used as a fixative for fabric dyes (pH indicator dyes bonded to cotton fabrics—dye retained after machine laundering, and also may be used in wearable pH indicator garments which are acid or base sensitive). It may be used as a preservative for foods, biological specimens, forensic specimens and biological specimens and lumber. It may be used as a buffer for noxious solutions or to inhibit the corrosive properties of bleaching solutions.

Further applications include etching aluminum or porcelain, and use as an anti-freeze or water purifier. Because the invention is compatible with pH indicator dyes, the solution efficacy can be visually determined. It is also noteworthy that the present solution may be reused (i.e. can be recycled).

Applications also include use of the invention as a replacement for conventional battery acid, in the process of pickling metals, in the process of tanning leather, to solubilize suspended solids (such as tats, lipids, etc.), and fixing fluorochemicals in the textile and related industries.

The invention may also be used as a pharmaceutical compound or therapeutic agent to be taken orally, parenterally, topically, or by any other route, and various other substances may be added to the core aqueous acidic composition as desired for this purpose. Pharmaceutical agents may include, but are not limited to, anesthetic agents, alcohols, creams, gels, aloe vera, vitamin E, PFP (polyfluorenated perfluorate (e.g. TEFLON® or FOMBLIN(K), moisturizers, emollients, surfactants, humectants, scents, colorants, glycerin, propylene glycol, emulsifiers, wetting agents, pH indicator dyes, medically-relevant dyes, contrasting agents, and carriers known in the art. Uses of the pharmaceutical agents or compounds formulated with the two-acid composition include, but are not limited to, deodorants, mouthwashes, topical antimicrobial ointment for wounds, and compositions for the treatment of a wide variety of maladies, including dry skin, wrinkles, acne, age spots, sunburn, infections (viral, bacterial and fungal), insect bites and rashes.

Such pharmaceutical agents may be appropriate for use on mucous membranes, including the mouth and eyes. For example, the present acid composition can be applied as a reactive topical skin protectant by mixing the two core-acids with a perfluorinted polyether grease vehicle, a water-based vehicle, or other suitable vehicle. Use of alternative topical delivery vehicles allows for flexibility in application as a topical protectant. The resulting decontamination/protection barrier is active over a broad temperature range of from about −10.degree. C. to about 50.degree. C., and is stable for one year or longer when stored at ambient temperatures. Alternatively, the pharmaceutical agent or compound can be brought into contact with the surface to be treated either directly or via applicators, including, but not limited to, sponges, towelettes and pads.

The invention may also be used as a decontamination agent useful in military and industrial applications to provide protection from, or directly inactivate, a variety of toxic chemical agents, such as those used in chemical warfare, farming, and lawn care. Such toxic chemicals include, but are not limited to, insecticides, pesticides, mustards, nerve agents, blister agents, cholinesterases and cholinesterase inhibitors in general. Additionally, decontamination agents according to the invention are effective in the inactivation of biologically toxic molecules such as those used in warfare. Biologically toxic molecules include, but are not limited to, aflatoxins, biological toxins, exotoxins, endotoxins, poisons, phytotoxins, insect and animal venoms and mycotoxins. Because of the non-caustic nature of the acid compositions of Yet another embodiment of the invention includes cleansing agents containing the acid composition. These cleansing agents include, but are not limited to, glass cleansers, metal cleansers, household cleaning solutions (kitchen and bathroom), and solutions to remove oxidation build-up from pipes and water heaters and heat exchangers. In this use of the invention, detergents, soaps, scents or strong acids may be added as needed to the acid composition. For use as a cleansing agent, the ratios of the core two-acid ingredients are preferably varied. Hydrochloric acid is added to the acid composition in ratios ranging from about 0.1 parts to 30 parts (by volume) of the acid composition to produce a solution suitable for use as a metal cleaner. Addition of more hydrochloric acid reduces the time required for cleaning, however, this may result in a product which irritates skin.

Another embodiment of the invention is a composition for processing food. Food processing compositions of the present invention may be suitable for decontaminating food items, such as, for example, meats, fruits or vegetables. In a preferred embodiment, the composition is applied to fruits and/or vegetables to remove or effectively destroy residual pesticides and microbes. The food processing composition may also be suitable for use as a de-odorizer for seafood and as an antimicrobial treatment for meat products.

The food processing compositions of the invention may be used as an antimicrobial process aid for the treatment of meat products such as poultry, beef, pork, game and seafood. In this context, the efficacy of present invention has been demonstrated as per the following examples.

### EXAMPLE 4

### Demonstration of Antimicrobial Activity (Salmonella Typhimurium)

The following table represents the final results from a study to determine the effectiveness against Salmonella Typhimurium. The microbiocide was mixed with phosphate buffered saline (PBS)(Treatment groups) until a pH of 1.5-1.6 was achieved. No microbiocide was added to the PBS in the control groups, and the pH of the control groups was between 5.8-6.0. Salmonella were inoculated into the tubes at 5 different concentrations: 10⁸, 10⁷, 10⁶, 10⁵, 10⁴. Samples were collected from each of the tubes 10 minutes after inoculating the samples with Salmonella and placed on blood agar plates. The plates were incubated at 37° C. for 24 hours under aerobic conditions. The treatment and control samples were also incubated for 24 hours at 37° C. Samples were again collected at 24 hours, placed on blood agar plates, and incubated using the methods described previously.

| Group | Inoculum | # post 10 min Rx | | # post 24 h Rx | |
|---|---|---|---|---|---|
| Treatment | 10⁸ | 0 | | 0 | |
| Treatment | 10⁸ | 100 | cfu/ml | 0 | |
| Treatment | 10⁸ | 0 | | 0 | |
| Control | 10⁸ | >1,000,000 | cfu/ml | >1,000,000 | cfu/ml |
| Control | 10⁸ | >1,000,000 | cfu/ml | >1,000,000 | cfu/ml |
| Treatment | 10⁷ | 100 | cfu/ml | 0 | |
| Treatment | 10⁷ | 400 | cfu/ml | 0 | |
| Treatment | 10⁷ | 0 | | 0 | |
| Control | 10⁷ | >1,000,000 | cfu/ml | >1,000,000 | cfu/ml |
| Control | 10⁷ | >1,000,000 | cfu/ml | >1,000,000 | cfu/ml |
| Treatment | 10⁶ | 0 | | 0 | |
| Treatment | 10⁶ | 0 | | 0 | |
| Treatment | 10⁶ | 0 | | 0 | |
| Control | 10⁶ | >100,000 | cfu/ml | >100,000 | cfu/ml |
| Control | 10⁶ | >100,000 | cfufml | >100,000 | cfu/mI |
| Treatment | 10⁵ | 0 | | 0 | |
| Treatment | 10⁵ | 0 | | 0 | |
| Treatment | 10⁵ | 0 | | 0 | |
| Control | 10⁵ | >30,000 | cfu/mi | >30,000 | cfu/ml |
| Control | 10⁵ | >20,000 | cfu/ml | >130,000 | cfulml |
| Treatment | 10⁴ | 0 | | 0 | |
| Treatment | 10⁴ | 0 | | 0 | |
| Treatment | 10⁴ | 0 | | 0 | |
| Control | 10⁴ | 4,700 | cfu/ml | 1.500 | cfu/m |
| Control | 10⁴ | 5,500 | cfu/ml | 4,800 | cfu/ml |

Another embodiment of the present invention may be used in the agricultural industry as an antimicrobial to treat row crops, vegetable crops, fruit crops, nut crops, vine crops, tree/forestry production, turf, golf landscape materials, container and field grown ornamentals and crops, greenhouse grown crops and ornamental production greenhouses, floral crops, hydroponics production, substrate production, post harvest applications for agricultural crops, ornamental and floral production for various bacterial, fungal and viral intections, blights and diseases. Such infestations, blights and diseases include but are not limited to various mildews. rot, rusts, smuts, wilts and mosaic diseases. Further agricultural applications include but Lire not limited to pH adjustment to soil to benefit mineral adsorption by the root systems, carbonate and bicarbonate conversion for the purpose of soil conditioning, as a defoliant aid for crops such as but not limited to cotton, potatoes, peanuts. etc. The the invention may be used alone or as a base for the formulation of other products such as but not limited to fertilizers, herbicides, insecticides, soil conditioners, etc. Applications methods would include but are not limited to spray, fog, mist, granular, drench, chemigation, fertigation, injection and aerial application.

The above formulations and applications are intended to merely illustrate the wide range of utility of the compositions described herein and are not intended to be an exhaustive listing of all possible formulations and uses of compositions according to the invention. Also, as the list of substances approved by the U.S. Food and Drug Administration to be Generally Regarded As Safe (GRAS) is revised, so will be the acids available for use in the compositions of the invention. As will be clear to those of skill in the art, compositions that are safe for human ingestion or contact are likewise safe for ingestion or contact by other animals.

Having now fully set forth a detailed example and certain modifications incorporating the concept underlying the present invention, various other modifications will obviously occur to those skilled in the art upon becoming familiar with said underlying concept. It is to be understood, therefore, that within the scope of the appended claims, the invention may be practiced otherwise than as specifically set forth herein.

1. A stabilized acidic solution for inhibiting microbial growth and controlling pH comprising essentially of from about 1 to about 60 percent of an acidic core composition including at least two acids, including a first inorganic acid and a second organic acid.

2. The stabilized acidic solution for inhibiting microbial growth and controlling pH according to claim 1, wherein said first inorganic acid comprises hydrochloric acid and said second organic acid comprises citric acid.

3. The stabilized acidic solution for inhibiting microbial growth and controlling pH according to claim 2, further comprising water.

4. The stabilized acidic solution for inhibiting microbial growth and controlling pH according to claim 3, wherein said hydrochloric acid is provided in a range of between 0.1 to about 10 weight percent of the total solution.

5. The stabilized acidic solution for inhibiting microbial growth and controlling pH according to claim 4, wherein said hydrochloric acid is provided in a range of between 0.5 to 5 weight percent of the total solution.

6. The stabilized acidic solution for inhibiting microbial growth and controlling pH according to claim 4, wherein said citric acid is provided in a range of between 4.95 to 50 weight percent of the total solution.

7. The stabilized acidic solution for inhibiting microbial growth and controlling pH according to claim 2, wherein said acid composition has a pH of 4 or less and is not corrosive to human skin.

8. The stabilized acidic solution for inhibiting microbial growth and controlling pH according to claim 1, wherein said first inorganic acid comprises sulfuric acid and said second organic acid comprises citric acid.

9. The stabilized acidic solution for inhibiting microbial growth and controlling pH according to claim 8, further comprising water.

10. The stabilized acidic solution for inhibiting microbial growth and controlling pH according to claim 8, wherein said sulfuric acid is provided in a range of between 0.01 to about 60 weight percent of the total solution.

11. The stabilized acidic solution for inhibiting microbial growth and controlling pH according to claim 10, wherein said citric acid is provided in a range of between 4.95 to 50 weight percent of the total solution.

12. The stabilized acidic solution for inhibiting microbial growth and controlling pH according to claim 11, wherein said acid composition has a pH of 4 or less and is not corrosive to human skin.

13. A method of preparing a stabilized acidic solution for inhibiting microbial growth and controlling pH, comprising the steps of:
adding a first acid composition consisting of one or more inorganic acids that dissociate nearly to completion in water; and
mixing a second organic acid in water to a final weight concentration of about 1 to 60%; and
mixing said first acid composition into said second acid composition such that the final composition consists from about 1 to 75 percent by weight of the second acid in the first acid composition.

14. The method of preparing a stabilized acidic solution for inhibiting microbial growth and controlling pH according to claim 13, wherein said first step of adding a first acid composition further comprises adding hydrochloric acid, and said second said second step of mixing a second organic acid in water further comprises mixing citric acid in water.

15. The method of preparing a stabilized acidic solution for inhibiting microbial growth and controlling pH according to claim 13, wherein said step of mixing said first acid composition into said second acid composition yields a stabilized acidic solution in which hydrochloric acid is present in a range of between 0.1 to about 10 weight percent of the total solution.

16. The method of preparing a stabilized acidic solution for inhibiting microbial growth and controlling pH according to claim 13, wherein said step of mixing said first acid composition into said second acid composition yields a stabilized acidic solution in which hydrochloric acid is present in a range of between 0.5 to 5 weight percent of the total solution.

17. The method of preparing a stabilized acidic solution for inhibiting microbial growth and controlling pH according to claim 15, wherein said step of mixing said first acid composition into said second acid composition yields a stabilized acidic solution in which citric acid is present in a range of between 4.95 to 50 weight percent of the total solution.

18. The method of preparing a stabilized acidic solution for inhibiting microbial growth and controlling pH according to claim 17, wherein said step of mixing said first acid composition into said second acid composition yields a stabilized acidic solution with a pH of 4 or less that is not corrosive to human skin.

19. The method of preparing a stabilized acidic solution for inhibiting microbial growth and controlling pH according to claim 13, wherein said first step of adding a first acid composition further comprises adding sulfuric acid, and said second said second step of mixing a second organic acid in water further comprises mixing citric acid in water.

20. The method of preparing a stabilized acidic solution for inhibiting microbial growth and controlling pH according to claim 19, wherein said step of mixing said first acid composition into said second acid composition yields a stabilized acidic solution in which sulfuric acid is present in a range of between 0.01 to about 60 weight percent of the total solution, and said citric acid is present in a range of between 4.95 to 50 weight percent of the total solution.
